(19) United States
(12) Patent Application Publication
(10) Pub. No.: US 2018/0000552 A1
(43) Pub. Date: Jan. 4, 2018

(54) MEDICAL DEVICE PATIENT-READY TRAY ASSEMBLY

(71) Applicant: George W. Young, Mundelein, IL (US)

(72) Inventor: George W. Young, Mundelein, IL (US)

(21) Appl. No.: 15/633,043

(22) Filed: Jun. 26, 2017

Related U.S. Application Data
(60) Provisional application No. 62/356,167, filed on Jun. 29, 2016.

(51) Int. Cl. A61B 50/33; A61B 50/30

(52) U.S. Cl. CPC A61B 50/33; A61B 2050/3008; A61B 2560/0431

(57) ABSTRACT

A tray and means for connecting multiple devices and components of an electronic medical devices prior to delivery to patients. The tray allows patient monitoring systems to be pre-assembled and made patient-ready. Patients with compromised health can easily unpack and set up the monitoring system. The tray also allows for the devices and components to be easily repacked for return shipping. The ease of patient setup and use of the medical devices thereby improves patient compliance with the monitoring regimen.

### CROSS-REFERENCE TO RELATED APPLICATION

This application claims the benefit of priority of U.S. provisional application No. 62/356,167, filed Jun. 29, 2016, the contents of which are herein incorporated by reference.

### BACKGROUND OF THE INVENTION

The present invention relates to medical diagnostic devices, and more particularly to shipping and storage trays for delivery of medical device systems to end users.

Remote patient monitoring involves the use of medical devices that record and transmit patient vital signs to a central monitoring station. The devices that record or capture the vital signs usually consist of a weight scale, pulse oximeter, blood pressure monitor, peak flow meter and blood glucose monitor. The data from the recording devices is transmitted to a communication device that sends the data to the central station. The recording devices transmit data to the communication device, either wirelessly or via physical wired connections.

Current monitoring systems are typically supplied as a master pack of multiple individual devices. The devices are produced by a number of different companies and individually packaged for distribution and sale. The individual device package also contains accessory components such as power cords, adapter plugs, connecting cables and instruction booklets. The accessory components are often individually packaged within the unit device package. The individually packaged devices that comprise the monitoring system are packed into a shipping box for distribution to the end user.

At the destination, the various individual devices are unpacked to prepare for assembly of the monitoring system. The accessory components are also unpacked. In addition to multiple device components, there are numerous packaging components and instruction sheets. Each device is assembled and connected to a power source as required. The monitoring devices are connected to the communication device to complete the system assembly and initial set up.

After the devices are unpacked, it is not clear to the patient or untrained caregiver as to how to interconnect the devices and components. Set-up instructions are complicated. Assembly is too complicated for untrained individuals and therefore, it is typically required that a trained technician travel to the point of use to interconnect the devices and complete the initial set up.

Additionally, if the devices are slated for subsequent return at the end of the monitoring period, repacking is not practical due to the large number of device and packaging components.

As can be seen, there is a need for a system and method for assisting patients and caregivers in configuring a patient monitoring system for use in the home healthcare setting.

### BRIEF DESCRIPTION OF THE DRAWINGS

FIG. 1 is a front perspective view of an embodiment of a tray according to aspects of the present invention.

FIG. 2 is a top plan view of the tray shown in an unfolded condition.

FIG. 3 is a perspective view of an alternative embodiment of a tray according to aspects of the present invention.

FIG. 4 is a top plan view of the alternative embodiment of FIG. 3, shown in an unfolded condition.

FIG. 5 is a perspective view of an alternate embodiment of the invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

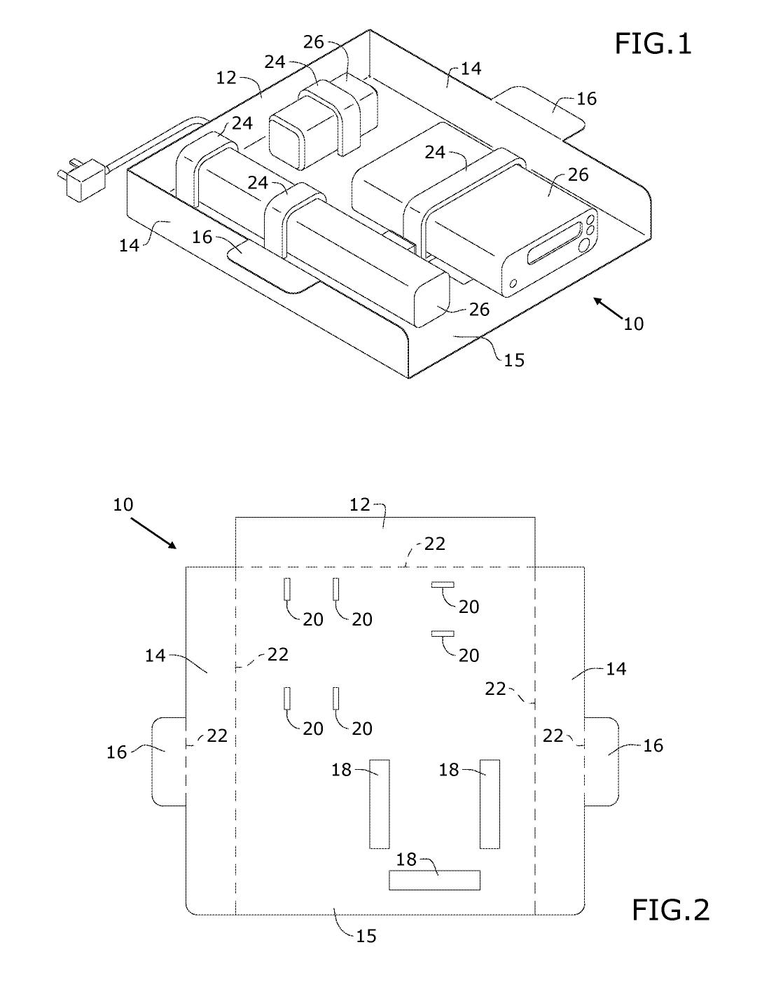

Broadly, embodiments of the present invention provide a system and method for packaging a medical monitoring system 26 in a configured condition for delivery to a patient or caregiver. As seen in reference to FIG. 1, the present invention includes a tray 10 that may be used to arrange the medical devices 26 and secure them for shipping in a preconfigured condition.

The tray 10 can include handles 16 to aid in the easy removal of the assembled tray 10 from the shipping box. The devices 26 are arranged in a manner that is efficient both for shipping and carrying in a pre-configured, patient-ready condition, ready for use by the patient without the need to interconnect the plurality of medical devices 26 before use. Devices 26 that have a physical and/or a wired connection are pre-connected and secured to one of a plurality of placement positions within the tray 10, such as via a securing means 24, such as a strap, twist tie, or the like, to prevent movement during shipping that could damage the devices 26 and interfere with the physical or wired connections of the devices 26.

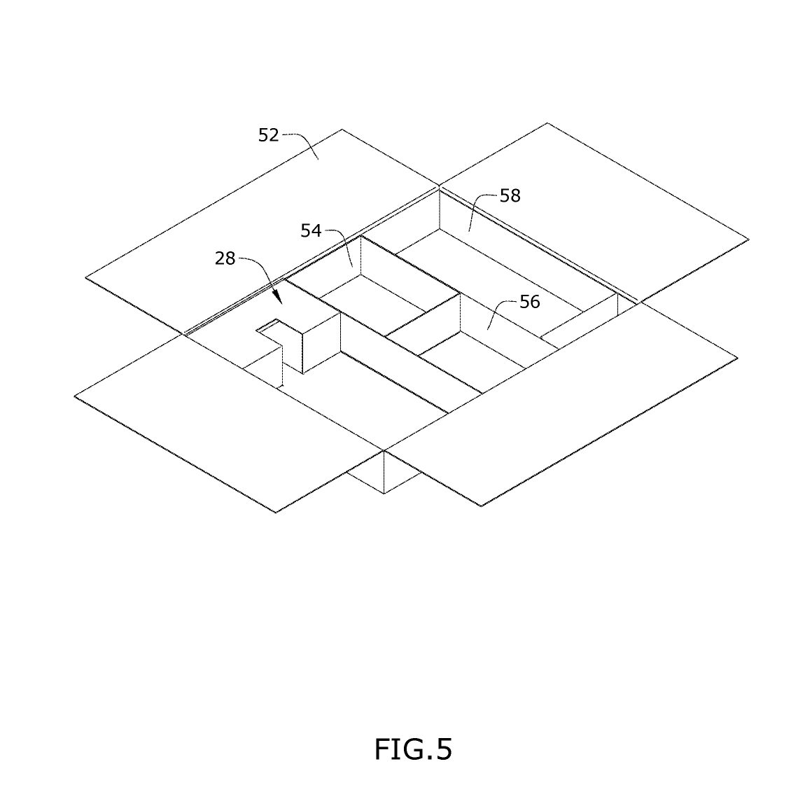

The tray 10 and method allows the untrained patient to remove the medical monitoring system 26 from the shipping box and easily connect to a power source and place the medical monitoring system 26 in use. The power source connection may be a common electrical plug that is configured to be inserted into a house electrical outlet, which is familiar to the patient. Each tray 10 may be custom designed for individual monitoring requirements and associated devices 26. As seen in reference to FIGS. 1 and 5, aspects of the invention can be accomplished by a single tray 10 or multiple trays 28, 54, 58, 56 containing different devices 26.

The tray 10 provides the means to assemble a ready-to-use medical monitoring system 26 prior to shipping to the patient. The component devices 26 are organized on the tray 10 for ease of use by the patient. Some devices 26 may be fastened to the tray 10 with straps or ties to prevent excessive movement during shipping. The assembled tray 10 is easily removed from the shipping box by the patient. The patient can easily locate the main power cord and plug it into a familiar conventional wall outlet. Instructions are simple and easy to follow. According to the present invention, a specialist is no longer required to be on-site with the patient to perform initial set up.

The tray 10 allows patient monitoring devices to be unpacked from manufacturer's containers and pre-assembled to make the system patient-ready. Patients with compromised health can easily unpack and set up the medical monitoring system. The tray 10 also allows for the medical monitoring system to be easily repacked for return shipping, such as when it is no longer needed for the patient's condition or for scheduled maintenance and calibration.

As seen in reference to FIGS. 1, a tray 10 allows pre-assembly of the medical monitoring system devices and components. The tray 10 may include a left and a right sidewall 14 that are integrally formed to the tray 10 and provide rigidity and containment of the devices 26 and components 26. The left and right sidewalls 14 may be formed along a score line 22.

The tray 10 includes a plurality of cut outs 18, 20 that are used to hold a device in place during shipping. The cut outs 18, 20 are adapted to receive a securing device 24, which may be straps or cable ties threaded through the cutouts 18, 20 to fasten devices 26 securely to the tray 10.

The tray 10 may include one or more lifting tabs 16 extending from the tray 10 sidewalls 14 to make it easy for the patient to remove the tray 10 from the shipping box. Preferably the tray 10 has a front opening 15 that allows the devices to remain in the tray 10 while the devices 26 are being used. The tray 10 may also be provided with a plurality of rubber feet attached to a bottom surface of the tray 10, preferably at the corners of the tray 10 to prevent the tray 10 from sliding when the device touch screen, or other controls may be operated by the user.

The tray 10 could be formed as a thermoformed tray 10, an assembled tray 10, or as shown in reference to FIGS. 2 and 4, formed from a die-cut sheet of cardboard, plastics or metal. The tray 10 could also be molded from polymer materials such as by thermoforming, injection molding or other molding processes. The tray 10 could also be assembled from multiple materials, such as polyethylene foam pieces glued to a paperboard sheet or other suitable material. In the case of a die-cut sheet, the tray 10 sides 14 may be positioned to about 90 degrees relative to the tray 10 bottom and secured in that position by an aft sidewall 12, also formed along score line 22 to provide rigidity to the tray 10 and the left and the right sidewalls 14. The aft sidewall 12 and the left and the right sidewalls may be joined by a suitable adhesive or a configuration of interlocking tabs. The rigidity of the tray 10 provides stability of the medical monitoring system 26 when the tray 10 is lifted out of the box or off a table.

The lifting tabs 16 on the sidewalls 24 of the tray 10 are used to grasp the tray 10 and lift it out of a shipping box. The outside dimensions of the tray 10 may be sized to fit snugly within a standard sized shipping box, which does not allow space for grasping both the inside and outside of the tray 10 wall 14 while in the shipping box to lift the tray 10 out of the box. The tabs 16 contribute to the ease of removing the tray 10 from the shipping box.

The open front 15 of the tray 10 allows a main communication device to be used while permitting the devices 26 to remain in the tray 10. The main communication device may, for example, include a touch screen device or may have buttons to operate or control the device 26. The open front 15 provides easy access to the controls of the main monitoring device 26. The rubber feet on the bottom of the tray 10 provide resistance to sliding when the tray 10 is placed on a table and the touch screen is activated or controls are pushed.

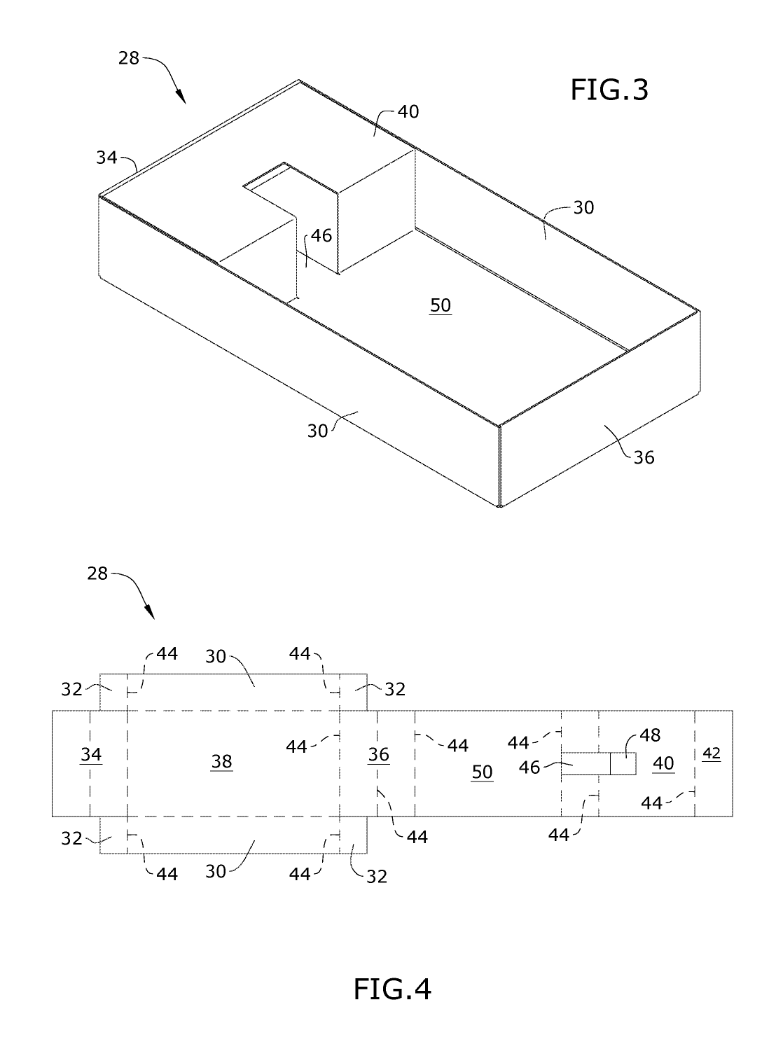

As seen in reference to FIGS. 3 & 4, an alternative embodiment of a tray 28 is shown. The tray has a left and a right upstanding sidewall 30, an aft sidewall 34, and a front sidewall 36, surrounding an interior bottom panel surface 50. An aft end of the tray 28 is configured with a housing 40 having an interior cavity and a notched access 46 in a top and an interior face of the housing 40. The notched access 46 allows pre-connected cables associated with the device 26 to be stored in a protected location. The notched access 46 allows limited side to side and end to end movement of a pre-connected device 26 such that the attached cable connector cannot contact the sides of the notched access 46 of the housing 40 to prevent damage to the device 26 during shipping. The notched access 46 may be of a plurality of different shapes. The sidewalls 30, 34, 36 are formed along a corresponding score line 44 for the respective panel. The front 36 and the aft 34 sidewalls may be formed as a double walled construction by an additional fold along score line 34, and 36, respectively. Similarly, the bottom may be formed as a double wall construction by having an exterior bottom panel 38.

The left and right 30, forward 36 and aft 34 sidewalls are configured as extensions of the exterior bottom panel 38. A plurality of tabs 32 may extend from the aft 34 and forward 36 sidewalls for joining with the left and the right sidewalls 30. A tab 48 may remain attached to the tray 28 or removed to extend the notched access 46 on the top of the housing 40. The housing 40 may be configured to be an open top cavity with the front panel of the housing 40 and notched access 46 remaining. The tray 28 may be assembled from a plurality of components.

As seen in reference to FIG. 5, the invention can also include using multiple trays 10. Components 26 or devices could be placed in separate trays 10 while being shipped in a connected condition. The trays 10 could also be stackable in or out of the shipping box. This approach would require less table space in the patient's home.

Additionally, the invention could be used in other than medical patient monitoring applications. It could be used where other devices are currently supplied and require unpacking, assembly and connection prior to use.

It should be understood, of course, that the foregoing relates to exemplary embodiments of the invention and that modifications may be made without departing from the spirit and scope of the invention as set forth in the following claims.

What is claimed is:

1. A carrier tray for shipping a plurality of medical devices in a pre-configured, patient-ready condition, comprising:
a tray having a mounting surface, a left and a right upstanding sidewall extending from lateral edges of the mounting surface interconnected by an aft sidewall upstanding from an aft edge of the tray;
a placement position defined on the mounting surface for each of the plurality of medical devices, the placement position defined between a plurality of cut outs extending through the mounting surface.

2. The carrier of claim 1, further comprising:
a fastener configured to be received through the plurality of cut outs and secure each of the plurality of medical devices in the placement position.

3. The carrier of claim 1, further comprising:
a handle extending laterally outward from a top edge of the left and the right upstanding sidewall for easy removal of the tray from a shipping box.

4. The carrier of claim 1, further comprising:
a front opening of the tray between the left and the right upstanding sidewall, configured to provide access to one or more operating controls of the plurality of medical devices.

5. The carrier of claim 1, further comprising:
a shipping carton having a bottom, a top and a plurality of sidewalls dimensioned to receive the tray.

6. A method for providing one or more medical monitoring devices to a designated patient in a pre-configured condition, comprising:
forming a tray comprising:
a mounting surface, a left and a right upstanding sidewall extending from lateral edges of the mounting surface interconnected by an aft sidewall upstanding from an aft edge of the tray; and
a placement position defined on the mounting surface for each of the one or more medical devices, the placement position defined between a plurality of cut outs extending through the mounting surface.

7. The method of claim 6, further comprising:
configuring the one or more medical monitoring devices for operational use by the designated patient.

8. The method of claim 7, further comprising:
placing the one or more medical monitoring devices on a corresponding placement position.

9. The method of claim 8, further comprising:
securing the one or more medical monitoring devices in the corresponding placement position with a fastener.

10. The method of claim 9, further comprising:
inserting the tray in a shipping container.

11. The method of claim 10, further comprising:
shipping the shipping container to the designated patient.

12. The method of claim 11, further comprising:
removing the tray from the shipping container.

13. The method of claim 12, further comprising:
monitoring the designated patient's medical conditions with the one or more medical monitoring devices.

14. A carrier tray for shipping a plurality of medical devices with one or more pre-connected cables, comprising:
a tray having a bottom panel, upstanding sidewalls and one or more internal compartments;
wherein, the one or more internal compartments are configured to limit movement of the plurality of medical devices when carried therein and are defined with a housing to receive the one or more pre-connected cables through an opening and to route the one or more pre-connected cables between the one or more internal compartments.
